(19) United States
(12) Patent Application Publication
(10) Pub. No.: US 2012/0059383 A1
(43) Pub. Date: Mar. 8, 2012

(54) INSTRUMENT FOR PLACEMENT AND IMPLANTATION OF A PROSTHETIC COMPONENT

(75) Inventors: Daniel P. Murphy, Warsaw, IN (US); Antony J. Lozier, Warsaw, IN (US); Samuel P. DeStefano, Fort Wayne, IN (US)

(73) Assignee: ZIMMER, INC., WARSAW, IN (US)

(21) Appl. No.: 13/186,155

(22) Filed: Jul. 19, 2011

Related U.S. Application Data
(60) Provisional application No. 61/379,964, filed on Sep. 3, 2010.

(51) Int. Cl. A61B 17/68

(52) U.S. Cl. 606/99

(57) ABSTRACT

The present disclosure provides a surgical instrument that creates a vacuum seal between a prosthetic component and a surgical instrument to grasp the prosthetic component so that the surgical instrument may be utilized to manipulate the prosthetic component relative to an anatomical structure. The surgical instrument is further designed to be used as an impactor capable of receiving and transferring an impaction force for seating the prosthetic component relative to the anatomical structure without the surgical instrument losing its grasp on the prosthetic component. The surgical instrument of the present disclosure includes a suction device which may be selectively released once the prosthetic component is appropriately positioned and seated relative to the anatomical structure. Such selective release prevents disruption of the positioning of the prosthetic component and facilitates proper placement and implantation of the prosthetic component.

### CROSS-REFERENCE TO RELATED APPLICATIONS

This application claims the benefit under 35 U.S.C. §119(e) to U.S. Provisional Patent Application Ser. No. 61/379,964, filed Sep. 3, 2010, the entire disclosure of which is hereby expressly incorporated by reference herein.

### BACKGROUND

1. Field of the Disclosure

This disclosure relates generally to an instrument used for orthopaedic surgery, and, more particularly, to an instrument for placement and implantation of a prosthetic component.

2. Description of the Related Art

During an orthopaedic procedure, prosthetic components can be positioned inside the body of a patient. To facilitate implantation of a prosthetic component, an incision is made in the body. Surgical instruments can then be used to grasp the prosthetic component and insert the prosthetic component through the incision made in the body while allowing for manipulation of the prosthetic component inside the body using a handle of the surgical instrument located outside of the body.

For example, reverse shoulder arthroplasty systems may include a ball-and-socket prosthesis in which a humeral component forms the socket portion of the joint and a glenoid component forms the ball portion of the joint. The glenoid component may include a hemispherical portion, or a glenoid head, which is attached to a glenoid base plate which is anchored in the glenoid of a patient. During an exemplary orthopaedic surgical procedure, the glenoid head is implanted onto the glenoid base plate in a patient. Once the glenoid head is positioned, the hemispherical surface provides an articulating surface for articulating with the humeral, or socket, portion of the reverse shoulder prosthesis. Correct placement and implantation of the glenoid head relative to the remainder of the reverse shoulder prosthesis facilitates proper functioning and longevity of the prosthesis.

### SUMMARY

The present disclosure provides a surgical instrument that creates a vacuum seal between a prosthetic component and a surgical instrument to grasp the prosthetic component so that the surgical instrument may be utilized to manipulate the prosthetic component relative to an anatomical structure. The surgical instrument is further designed to be used as an impactor capable of receiving and transferring an impaction force for seating the prosthetic component relative to the anatomical structure without the surgical instrument losing its grasp on the prosthetic component. The surgical instrument of the present disclosure includes a suction device which may be selectively released once the prosthetic component is appropriately positioned and seated relative to the anatomical structure. Such selective release prevents disruption of the positioning of the prosthetic component and facilitates proper placement and implantation of the prosthetic component.

The present disclosure, in one form thereof, comprises a surgical instrument for securement to a prosthetic component so that the surgical instrument may be utilized to manipulate the prosthetic component relative to an anatomical structure. The surgical instrument of this form of the present disclosure includes a prosthetic component contacting end sized and shaped relative to the prosthetic component such that the prosthetic component contacting end is capable of forming a continuous seal between the surgical instrument and the prosthetic component, an outer housing having an outer housing proximal end, an outer housing distal end, and a wall defining an elongate aperture between the outer housing proximal end and the outer housing distal end, the elongate aperture in fluid communication with the prosthetic component contacting end, a plunger having an outer periphery and a plunger central axis, the plunger sized for movement within the elongate aperture of the outer housing, a seal interposed between the plunger and the wall of the outer housing, the seal sized relative to the plunger and the outer housing so that, with the prosthetic component contacting end forming the continuous seal with the prosthetic component, movement of the plunger within the elongate aperture of the outer housing away from the prosthetic component contacting end effectuates a vacuum seal between the prosthetic component contacting end and the prosthetic component, and a lock connected to the outer housing and the plunger, the lock operable to selectively lock the plunger relative to the outer housing so that movement of the plunger within the elongate aperture of the outer housing toward the prosthetic component is prevented, whereby, with the lock locking the plunger relative to the outer housing so that movement of the plunger within the elongate aperture of the outer housing toward the prosthetic component is prevented, a force imparted to the plunger is transmitted to the prosthetic component contacting end via the lock and the outer housing.

The present disclosure, in another form thereof, comprises a method of implanting a prosthetic component, the method including: obtaining a surgical instrument, the surgical instrument including an active vacuum source; and a prosthetic component contacting end sized and shaped relative to the prosthetic component such that the prosthetic component contacting end is capable of forming a continuous seal between the surgical instrument and the prosthetic component; contacting the prosthetic component with the prosthetic component contacting end such that the continuous seal is formed; actuating the active vacuum source to effectuate a vacuum seal between the prosthetic component contacting end of the surgical instrument and the prosthetic component to secure the prosthetic component to the surgical instrument; positioning the prosthetic component, using the surgical instrument, until a desired position is achieved; and imparting an impaction force to the prosthetic component through the surgical instrument.

The present disclosure, in a further form thereof, comprises a method of implanting a prosthetic component, the method including: obtaining a surgical instrument, the surgical instrument including a prosthetic component contacting end; an outer housing having an outer housing proximal end, an outer housing distal end, and a wall defining an elongate aperture between the outer housing proximal end and the outer housing distal end, the elongate aperture in fluid communication with the prosthetic component contacting end; a plunger having an outer periphery, the plunger sized for movement within the elongate aperture of the outer housing; and a seal interposed between the plunger and the wall of the outer housing; contacting the prosthetic component with the prosthetic component contacting end such that a continuous seal is formed; after the step of contacting the prosthetic component, moving the plunger within the elongate aperture of the outer housing away from the prosthetic component contacting end to effectuate a vacuum seal between the prosthetic component contacting end of the surgical instrument and the prosthetic component to secure the prosthetic component to the surgical instrument; positioning the prosthetic component, using the surgical instrument, until a desired position is achieved; and disengaging the surgical instrument from the prosthetic component by breaking the vacuum seal.

### BRIEF DESCRIPTION OF THE DRAWINGS

The above-mentioned and other features and advantages of this disclosure, and the manner of attaining them, will become more apparent and the disclosure itself will be better understood by reference to the following description of embodiments of the disclosure taken in conjunction with the accompanying drawings, wherein:

FIG. 1 is an exploded, perspective view of a surgical instrument in accordance with the present disclosure;

FIG. 2 is an assembled, perspective view of the surgical instrument of FIG. 1 in a first position and a glenoid head;

FIG. 3 is an assembled, elevational view of the surgical instrument of FIG. 1 in a second position, with the surgical instrument secured to the glenoid head of FIG. 2;

FIG. 4 is an assembled, perspective view of the surgical instrument of FIG. 1 in a third, locked position, with the surgical instrument secured to the glenoid head of FIG. 2;

FIG. 5 is an assembled, perspective view of the surgical instrument of FIG. 1 in the third, locked position, with the surgical instrument secured to a glenoid head, and the surgical instrument and the glenoid head positioned adjacent a glenoid base plate secured to a glenoid;

FIG. 6 is an assembled, elevational view of the surgical instrument of FIG. 1 in a fourth position, with the surgical instrument removed from the glenoid head of FIG. 5 after implanting the glenoid head relative to the glenoid;

FIG. 7A is a cross-sectional view taken along line 7A-7A of FIG. 3;

FIG. 7B is a cross-sectional view taken along line 7B-7B of FIG. 6;

FIG. 8 is a fragmentary, cross-sectional view taken along line 8-8 of FIG. 4;

FIG. 9 is a fragmentary, cross-sectional view taken along line 9-9 of FIG. 6;

FIG. 10 is an exploded, perspective view of a surgical instrument in accordance with another exemplary embodiment of the present disclosure;

FIG. 11 is an assembled, perspective view of a surgical instrument in accordance with another exemplary embodiment of the present disclosure and a glenoid head; and

FIG. 12 is a cross-sectional view taken along line 12-12 of FIG. 11.

Corresponding reference characters indicate corresponding parts throughout the several views. The exemplifications set out herein illustrate exemplary embodiments of the disclosure, and such exemplifications are not to be construed as limiting the scope of the disclosure in any manner.

### DETAILED DESCRIPTION

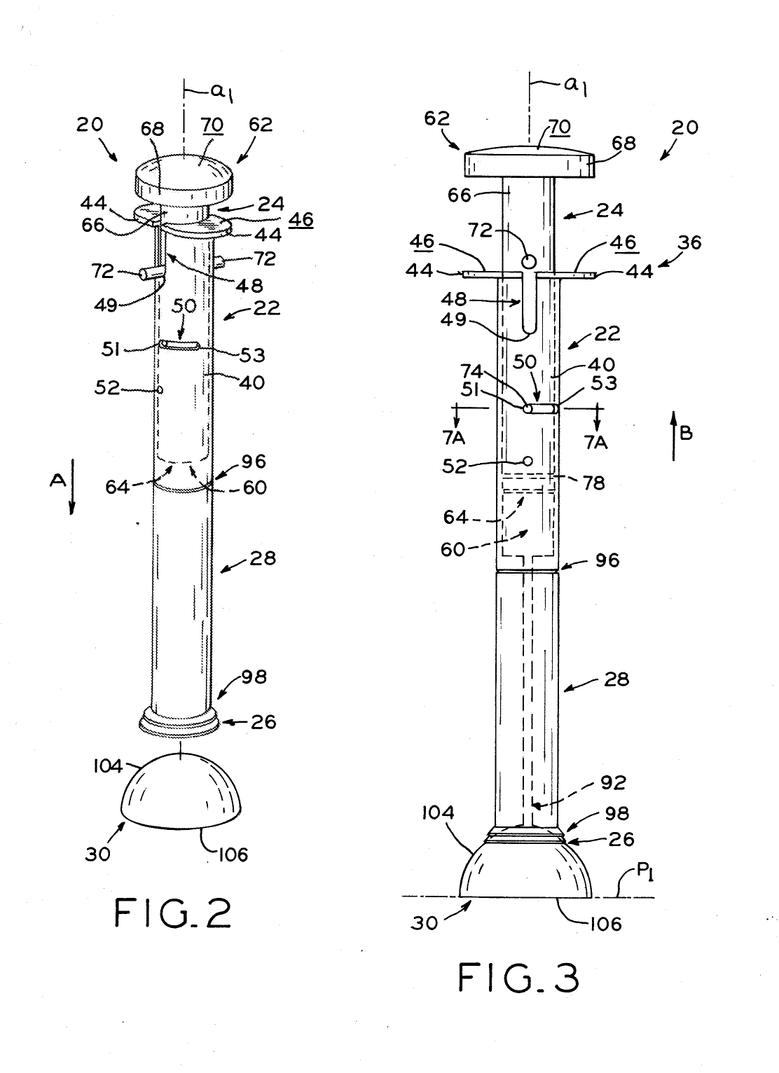

An exemplary surgical instrument in accordance with the present disclosure may be used to secure glenoid head 30, as illustrated in FIGS. 2-6, 8 and 9 and discussed below, or other similar prosthetic component to an anatomical structure, while maintaining the correct alignment and permitting impaction of the prosthetic component with a single surgical instrument. Although exemplified as a glenoid head inserter used in reverse shoulder arthroplasty, a surgical instrument in accordance with the present disclosure may also be used in conjunction with other prosthetic components such as, but not limited to, acetabular shells/liners, femoral knee implants, tibial knee implants, humeral heads, humeral liners, glenoid liners, and any other orthopaedic implants.

In the following discussion, “proximal” refers to a direction generally toward the heart of a patient, and “distal” refers to the opposite direction of proximal, i.e., away from the heart of a patient.

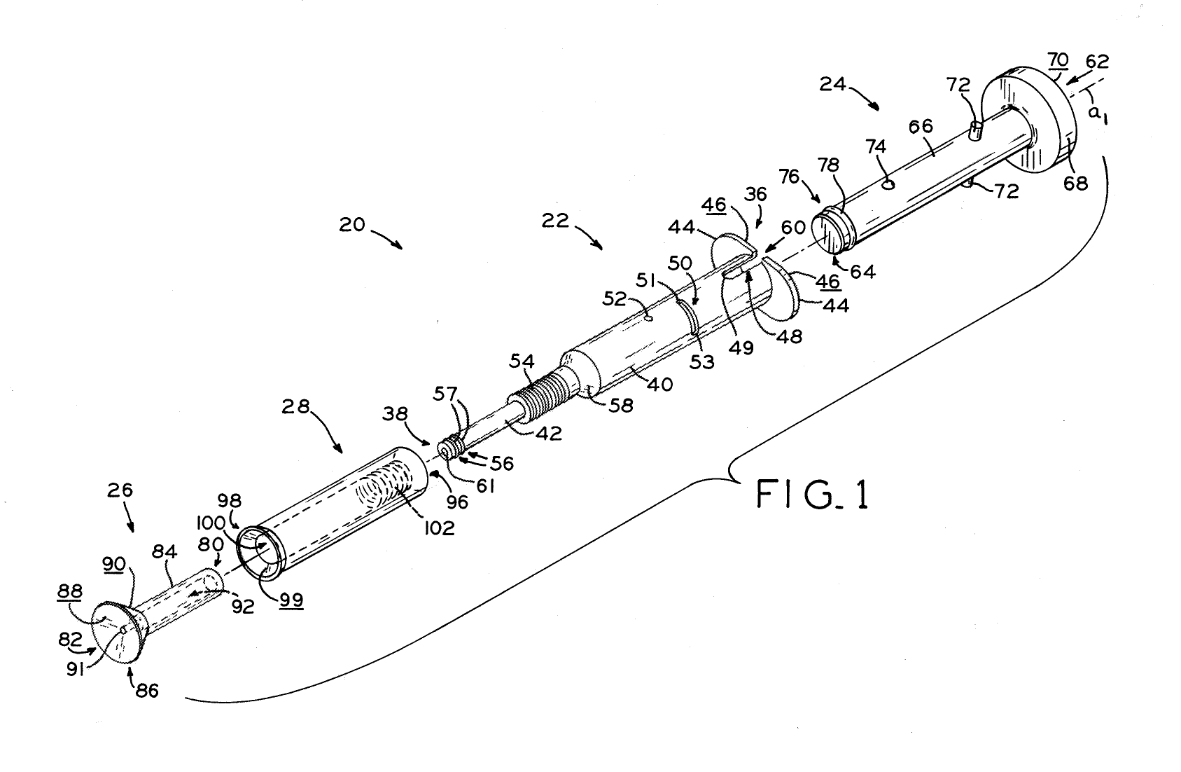

Referring to FIG. 1, surgical instrument 20 includes outer housing 22, plunger 24, suction cup 26, and sleeve 28. Surgical instrument 20 may be used to insert one of a plurality of different sized glenoid heads such as glenoid head 30 (shown in FIGS. 2-6) onto glenoid base plate 32 (shown in FIG. 5), attached to scapula 34 (shown in FIG. 5), of a reverse shoulder prosthesis assembly. For example, a surgical instrument of the present disclosure may be used with a reverse shoulder prosthesis assembly in accordance with the reverse shoulder system described in two brochures published by Zimmer, Inc., namely the “Zimmer® Trabecular Metal™ Reverse Shoulder System, Surgical Technique” brochure, copyright 2006, 2008 and 2010, and the “Anatomical Shoulder™ Inverse/Reverse, Surgical Technique” brochure, copyright 2010, the entire disclosures of which are hereby expressly incorporated herein by reference.

Surgical instrument 20 utilizes active suction to securely hold glenoid head 30 while transporting glenoid head 30 from a surgical tray to a surgical site and onto glenoid base plate 32 in an anatomical structure. Active suction differs from passive suction in that the active suction provided by surgical instrument 20 is controlled by physically actuating plunger 24 relative to outer housing 22 to create a vacuum seal between suction cup 26 and glenoid head 30. As opposed to passive suction which is created by pressing a suction cup against a flat surface. Advantageously, the active suction created using the surgical instrument in accordance with the present disclosure provides greater control to a user to select when to utilize the vacuum seal and when to disengage the vacuum seal. The force of the vacuum seal created by the surgical instrument is sufficient to hold and manipulate the prosthetic component during an orthopaedic procedure. By utilizing active suction, surgical instrument 20 is capable of securely holding glenoid head 30 for a desired duration of time and a surgeon can activate the vacuum seal once suction cup 26 of surgical instrument 20 is properly aligned to glenoid head 30. Also, the surgeon can select when to release the vacuum seal to disengage the surgical instrument 20 from glenoid head 30 without interacting directly with the portion of the instrument directly connected to the implant.

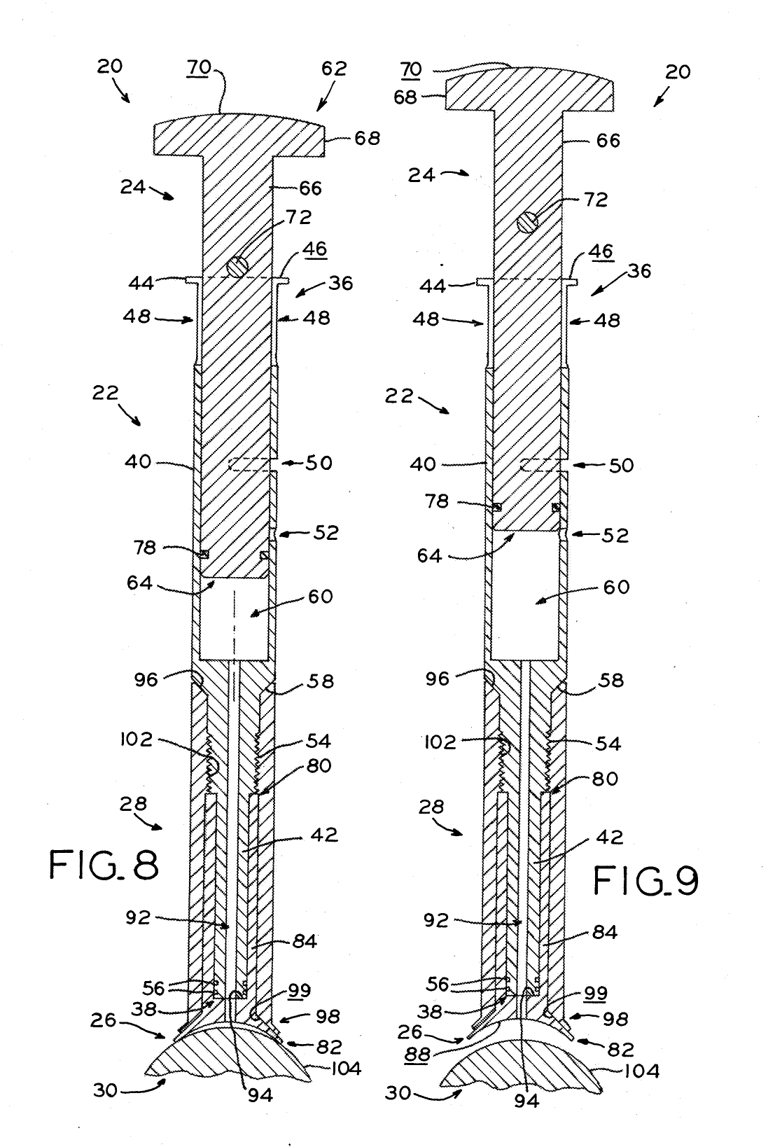

Referring to FIG. 1, outer housing 22 generally includes outer housing distal end 36, opposing outer housing proximal end 38, outer housing cylindrical body 40 located adjacent to distal end 36, and outer housing extending arm 42 located adjacent to proximal end 38. Outer housing distal end 36 includes flanges 44 extending substantially perpendicularly from distal end 36 of body 40. Flanges 44 define flange exterior surface 46. Outer housing cylindrical body 40 includes longitudinal slots 48 (both slots 48 shown in FIGS. 8 and 9) extending longitudinally from distal end 36 and terminating at end walls 49, circumferential slot 50 extending partially around a circumference of cylindrical body 40 and having first end wall 51 and opposing, second end wall 53, and air release aperture 52 formed therethrough body 40. In one embodiment, as illustrated in FIG. 1, circumferential slot 50 extends along 90 degrees of body 40 of outer housing 22. Outer housing extending arm 42 includes threaded portion 54 and at least one annular groove 56 adjacent to proximal end 38. A sealing mechanism such as o-ring 57 can be positioned within annular groove 56. Cylindrical body 40 and outer housing extending arm 42 meet at annular shoulder 58. The walls of cylindrical body 40 and extending arm 42 each define elongate aperture or outer housing cavity 60 of outer housing 22 respectively therein. Outer housing cavity 60 spans the extent of outer housing cylindrical body 40 and the extent of outer housing extending arm 42 so that outer housing 22 is cannulated along its entire length. Outer housing cavity 60 includes outer housing cavity opening 61 located at proximal end 38 and an opposing opening at distal end 36.

Referring to FIG. 1, plunger 24, which is sized for movement within cavity 60 of outer housing 22, generally includes plunger distal end 62, opposing plunger proximal end 64, plunger cylindrical body 66, and plunger impaction head 68 located at distal end 62. Impaction head 68 defines impaction surface 70. Impaction surface 70 is capable of receiving and transferring an impaction load for seating glenoid head 30 onto glenoid base plate 32. As discussed in more detail later, after the vacuum seal has been activated to secure glenoid head 30 to suction cup 26, surgical instrument 20 is placed in a locked, third position, and glenoid head 30 is properly aligned relative to glenoid base plate 32, impaction surface 70 of surgical instrument 20 can be impacted using a surgical tool such as a surgical mallet to properly seat glenoid head 30 onto glenoid base plate 32. Because surgical instrument 20 is capable of being impacted via impaction surface 70, the need for a separate impaction instrument is eliminated. Because surgical instrument 20 can be positioned in a locked position, as will be discussed in more detail below, impact loads can be applied through surgical instrument 20 to glenoid head 30 without surgical instrument 20 losing its grasp on glenoid head 30.

Referring to FIG. 1, plunger cylindrical body 66 of plunger 24 includes cross-pin 72 extending from opposing sides of body 66. Plunger cylindrical body 66 also includes ball detent 74. In alternative embodiments, plunger cylindrical body 66 may include a spring loaded locking mechanism, latch, or other similar mechanism. Referring to FIGS. 7A and 7B, body 66 includes ball detent cavity 75 therein which receives ball detent 74, spring 77, and retention member 79. Ball detent cavity 75 comprises a passageway sized to allow movement of ball detent 74 therein and terminating in an opening through which ball detent 74 extends beyond the periphery of cylindrical body 66. The opening at which ball detent cavity 75 terminates is smaller than the diameter of ball detent 74 so that ball detent 74 cannot exit ball detent cavity 75. The opening at which ball detent cavity 75 terminates may be elastically deformable to allow initial loading of ball detent 74. Alternatively, ball detent 74 may be initially loaded into a first end of ball detent cavity 75. Next, spring 77 and retention member 79 may be loaded into the first end of ball detent cavity 75 so that spring 77 is connected to and disposed between ball detent 74 and retention member 79.

With plunger 24 positioned within outer housing cylindrical body 40, the interior walls of outer housing cylindrical body 40 depress ball detent 74 within ball detent cavity 75 via compression of spring 77. Referring to FIG. 3, once ball detent 74 is aligned with circumferential slot 50 of outer housing 22, spring 77 forces ball detent 74 into engagement with circumferential slot 50. Plunger cylindrical body 66 also includes annular groove 76 located adjacent proximal end 64 for receiving a sealing mechanism such as o-ring 78. In another embodiment, the sealing mechanism interposed between plunger 24 and outer housing 22 may be a diaphragm. A diaphragm could be moved from a position toward suction cup 26 to a position away from suction cup 26. In other embodiments, the sealing mechanism may be a singular o-ring or a plurality of o-rings, a diaphragm, or a flexible polymer cap composed of an elastomer, rubber, or other material with similar mechanical and physical properties, such as a similar durometer rating.

Referring to FIG. 1, suction cup 26 defines a prosthetic component contacting end and generally includes suction cup distal end 80, opposing suction cup proximal end 82, suction cup body 84, and suction cup head 86. Suction cup head 86 includes suction surface 88 and opposing suction head exterior surface 90. Suction cup cavity 92 extends through suction cup 26 from proximal end 82 to distal end 80. Suction cup cavity 92 includes suction cup cavity opening 91 located therein suction head surface 88. An interior surface of suction cup head 86 also defines suction cup shoulder 94 (shown in FIGS. 8 and 9). Suction cup 26 is sized and shaped relative to a prosthetic component such as glenoid head 30 such that suction cup 26 is capable of forming a continuous seal, i.e., a seal sufficiently tight so that any air that may escape is not enough to break the vacuum seal (further described below) between surgical instrument 20 and a prosthetic component during a normal surgical procedure, between surgical instrument 20 and glenoid head 30.

Referring to FIG. 1, sleeve 28 generally includes sleeve distal end 96, opposing sleeve proximal end 98, and annular sleeve proximal surface 99. Sleeve cavity 100 extends through sleeve 28 from distal end 96 to proximal end 98. Sleeve cavity 100 also includes threaded portion 102. Annular sleeve proximal surface 99 of sleeve 28 supports suction cup 26.

Referring to FIGS. 1 and 2, to assemble surgical instrument 20 a user, e.g., a surgical technician, nurse, or surgeon, inserts proximal end 64 of plunger 24 into cavity 60 of outer housing cylindrical body 40 such that cross-pin 72 of plunger 24 is received within respective longitudinal slots 48 of outer housing 22. Referring to FIG. 2, plunger 24 is inserted into cavity 60 of outer housing cylindrical body 40 until cross-pin 72 contacts respective end walls 49 of longitudinal slots 48. The position illustrated in FIG. 2 corresponds to an initial or first position of plunger 24 relative to outer housing 22.

Plunger 24 cooperates with outer housing 22 such that plunger 24 can axially move relative to outer housing 22 along longitudinal axis a₁ (shown in FIGS. 1-3). The sealing mechanism interposed between plunger 24 and outer housing 22 provides a sealed relationship between plunger 24 and outer housing 22. The sealing mechanism interposed between plunger 24 and outer housing 22 is sized relative to plunger 24 and outer housing 22 so that, with suction cup 26 forming the continuous seal with a prosthetic component such as glenoid head 30, movement of plunger 24 within cavity 60 of outer housing 22 away from suction cup 26 effectuates a vacuum seal between suction cup 26 and the prosthetic component.

In one embodiment, sleeve 28 is next attached to outer housing proximal end 38. Referring to FIGS. 1, 8 and 9, sleeve 28 is slid over outer housing extending arm 42 until threaded portion 102 of sleeve 28 receives threaded portion 54 of extending arm 42. Sleeve 28 is then threaded on extending arm 42 until sleeve distal end 96 abuts annular shoulder 58 of outer housing 22. As illustrated in FIGS. 8 and 9, in an embodiment of surgical instrument 20 including sleeve 28, sleeve 28 must be attached to outer housing proximal end 38 before attaching suction cup 26 to outer housing proximal end 38. Once sleeve 28 is threadingly connected to extending arm 42, suction cup 26 is positioned over outer housing proximal end 38. Referring to FIGS. 1, 8 and 9, suction cup body 84 is positioned within sleeve cavity 100 and attached to outer housing proximal end 38 such that outer housing extending arm 42 is received within suction cup cavity 92. In this embodiment, referring to FIGS. 1, 8 and 9, sleeve 28 is located over suction cup body 84 such that annular sleeve proximal surface 99 abuts suction head exterior surface 90. Sleeve 28 provides support to suction cup head 86 and increases the stabilization between suction cup 26 and glenoid head 30 when surgical instrument 20 is secured to glenoid head 30. The increased support provided by sleeve 28 to suction cup 26 also allows suction cup 26 of surgical instrument 20 to maintain sufficient rigidity when inserted into a surgical site such that surgical instrument 20 is capable of retracting tissues, i.e., drawing back tissues, in limited access conditions in a surgical site.

In one embodiment, to further secure suction cup 26 to extending arm 42 of outer housing 22, at least one sealing mechanism such as o-ring 57 is seated in annular groove 56 before positioning suction cup body 84 over outer housing extending arm 42. Subsequently, as suction cup body 84 is positioned over outer housing extending arm 42, o-ring 57 is compressed between annular groove 56 of extending arm 42 and the walls of suction cup body 84 defining suction cup cavity 92, thereby further sealing extending arm 42 to suction cup body 84.

In an alternative embodiment, sleeve 28 is not utilized and suction cup 26 is attached to outer housing proximal end 38. Referring to FIG. 1, suction cup body 84 is positioned over outer housing extending arm 42 such that outer housing extending arm 42 is received within suction cup cavity 92. Suction cup 26 is secured to extending arm 42 of outer housing 22 via friction-fit engagement between the walls of suction cup body 84 defining suction cup cavity 92 and outer housing extending arm 42. As shown in FIG. 8, suction cup 26 is slid over extending arm 42 of outer housing 22 until suction cup distal end 80 abuts the proximal end of threaded portion 54 of outer housing extending arm 42 and proximal end 38 of outer housing extending arm 42 abuts suction cup shoulder 94. In embodiments in which sleeve 28 is not utilized, extending arm 42 of outer housing 22 may not include threaded portion 54.

Referring to FIGS. 3, 6, 8 and 9, with surgical instrument 20 assembled, outer housing cavity 60 and suction cup cavity 92 are in fluid communication, i.e., air is capable of flowing from suction cup cavity opening 91 through suction cup cavity 92, outer housing cavity opening 61, outer housing cavity 60, and out distal end 36 of outer housing 22. Referring to FIGS. 1-3, in an exemplary embodiment, plunger 24 has a plunger central axis aligned along longitudinal axis a₁, outer housing 22 has an outer housing central axis aligned along longitudinal axis a₁, and suction cup 26 has a suction cup central axis aligned along longitudinal axis a₁. In such an embodiment, the suction cup central axis is aligned with the outer housing central axis and the plunger central axis. Further, sleeve 28 has a sleeve central axis aligned along longitudinal axis a₁. In such an embodiment, the sleeve central axis is aligned with the suction cup central axis, the outer housing central axis, and the plunger central axis.

Outer housing 22, plunger 24, and sleeve 28 of surgical instrument 20 can each be made of biocompatible surgical grade materials such as stainless steel. In certain embodiments, outer housing 22, plunger 24, and sleeve 28 of the present disclosure will each be able to be sterilized and reused after a first use. Outer housing 22, plunger 24, and sleeve 28 of surgical instrument 20 can also each be made of various durable plastics such as polyethylene, polyphenylsulfone (Radel), polyetheretherketone (PEEK), polyetherimide (Ultem), or other polymers with similar mechanical and physical properties. In certain embodiments, outer housing 22, plunger 24, and sleeve 28 of the present disclosure will each be able to be disposable after a single use.

Referring to FIGS. 2-5, glenoid head 30 generally includes spherical exterior wall or articulating surface 104, planar end wall 106 defining plane p₁ (FIG. 3), and peg 108 (shown only in FIG. 5) extending perpendicularly from planar end wall 106. Referring to FIG. 5, as previously discussed, in a reverse shoulder system glenoid head 30 is attached to glenoid head contacting surface 109 of glenoid base plate 32 anchored in scapula 34 of a patient.

Referring to FIGS. 2-6, the use of surgical instrument 20 to align and place glenoid head 30 to glenoid base plate 32 will now be described. Referring to FIG. 2, a user such as a surgeon obtains a surgical instrument in accordance with the present disclosure for inserting a glenoid head onto a glenoid base plate of a reverse shoulder prosthesis assembly. The surgeon initially inserts plunger 24 into cavity 60 of outer housing cylindrical body 40, such that cross-pin 72 of plunger 24 is positioned within longitudinal slots 48 of outer housing 22, until cross-pin 72 of plunger 24 contacts respective end walls 49 of longitudinal slots 48. This position, as illustrated in FIG. 2, corresponds to the initial or first position of plunger 24 relative to outer housing 22.

Next, surgical instrument 20 can be positioned relative to glenoid head 30. Referring to FIGS. 1-3, because outer housing 22, plunger 24, suction cup 26, and sleeve 28 are all aligned along a single central axis, i.e., longitudinal axis a₁, the surgeon has the ability to visually align the central axis of surgical instrument 20 ninety degrees relative to plane p₁ (FIG. 3) including planar end wall 106 of glenoid head 30 in an open environment such as on a surgical tray.

Referring to FIG. 2, once suction cup 26 of surgical instrument 20 is properly aligned with articulating surface 104 of glenoid head 30 and surgical instrument 20 is in the first position as shown in FIG. 2, a user releasably attaches glenoid head 30 to surgical instrument 20 by applying force in a direction generally along arrow A (FIG. 2) to force suction cup 26 against glenoid head 30 and form a continuous seal between suction cup 26 and glenoid head 30. In this position, air release aperture 52 is sufficient sealed, i.e., the sealing mechanism such as o-ring 78 located adjacent proximal end 64 of plunger 24 is positioned between air release aperture 52 and suction cup 26 so that air is prevented from entering outer housing cavity 60 via air release aperture 52. Applying force in a direction generally along arrow A (FIG. 2) to force suction cup 26 against glenoid head 30 allows surgical instrument 20 to also be used as a passive suction device. In an alternative embodiment, suction cup 26, and the structures of outer housing 22 and plunger 24 for engaging and assembling with suction cup 26, may be formed substantially similar to the structures described in U.S. patent application Ser. No. 12/783,971, filed May 20, 2010, entitled “Introduction Instrument,” the entire disclosure of which is hereby expressly incorporated herein by reference.

Referring now to FIG. 3, to provide the vacuum seal between surgical instrument 20 and glenoid head 30, the user slowly moves plunger 24 along longitudinal axis a₁, i.e., in the direction of arrow B, to create a vacuum seal between suction cup 26 and glenoid head 30. In this manner, suction cup 26 of surgical instrument 20 is secured to articulating surface 104 of glenoid head 30. In one embodiment, the user slowly moves plunger 24 along longitudinal axis a₁, i.e., in the direction of arrow B, from the first position (FIG. 2) to the second position (FIG. 3) relative to outer housing 22, i.e., until ball detent 74 located on plunger cylindrical body 66 traverses into circumferential slot 50 of outer housing 22. The engagement of cross-pin 72 of plunger 24 in longitudinal slots 48 of outer housing 22 provides proper alignment of plunger 24 relative to outer housing 22 and facilitates ball detent 74 engaging circumferential slot 50. In one embodiment, ball detent 74 may function as the mechanism which defines the starting, locked, and air release positions of surgical instrument 20. In alternative embodiments, the locking components, i.e., cross-pin 72 engaged with longitudinal slots 48 and ball detent 74 engaged with circumferential slot 50, can be respectively located on either outer housing 22 or plunger 24. In other embodiments, other locking components with a similar function can be used to control movement of plunger 24 relative to outer housing 22.

This movement of plunger 24 from the first position (FIG. 2) relative to outer housing 22 to the second position (FIG. 3) relative to outer housing 22 in a direction along arrow B (FIG. 3) creates the vacuum seal between surgical instrument 20 and glenoid head 30. As the user slowly moves plunger 24 from the first position (FIG. 2) to the second position (FIG. 3), the user actively increases the volume within outer housing cavity 60. Because the sealing mechanism interposed between plunger 24 and outer housing 22 does not allow any air to enter into outer housing cavity 60 and suction cup 26 is engaged in a continuous seal with glenoid head 30 such that no air can enter into suction cup cavity 92, the same number of air molecules are located within outer housing cavity 60 as the user actively increases the volume within outer housing cavity 60. This decreases the pressure in outer housing cavity 60 relative to the air pressure outside of surgical instrument 20. Therefore, a vacuum, i.e., a space of lower air pressure, is created to secure suction cup 26 to glenoid head 30.

The vacuum seal of the present disclosure creates active suction which is controlled by the user, thereby providing a controlled, secure connection between surgical instrument 20 and glenoid head 30. The force of the vacuum seal created by surgical instrument 20 is sufficient to hold and manipulate the prosthetic component such as glenoid head 30 during an orthopaedic procedure. The active suction also allows the user the ability to select when to utilize the vacuum seal and when to disengage the vacuum seal once proper alignment and placement of glenoid head 30 relative to glenoid base plate 32 is achieved.

Referring to FIG. 3, with plunger 24 in the second position (FIG. 3) relative to outer housing 22, cross-pin 72 is no longer engaged with longitudinal slot 48 and cross-pin 72 is located relative to outer housing 22 at a position beyond flange exterior surface 46 of outer housing 22, thereby allowing plunger 24 to rotate with respect to outer housing 22. Also, with plunger 24 in the second position (FIG. 3), ball detent 74 is engaged with circumferential slot 50 such that ball detent 74 is positioned adjacent first end wall 51 of slot 50.

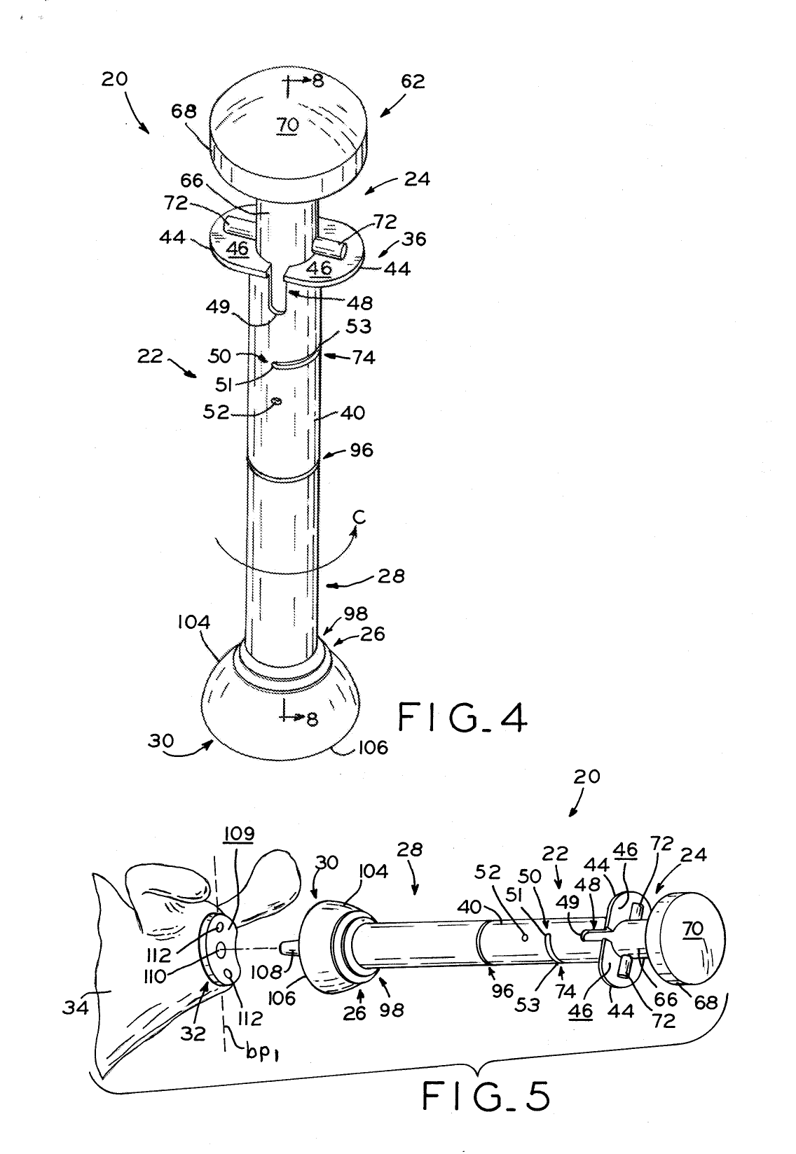

Referring to FIGS. 3 and 4, once the vacuum seal is created and glenoid head 30 is secured to suction cup 26, the user can now rotate plunger 24 counter-clockwise, i.e., in the direction of arrow C (FIG. 4), approximately 90 degrees from the second position (FIG. 3) relative to outer housing 22 to a third position (FIG. 4) relative to outer housing 22. Rotation of plunger 24 from the second position (FIG. 3) to the third position (FIG. 4) is guided by the engagement of ball detent 74 with circumferential slot 50. For example, second end wall 53 of circumferential slot 50 provides a physical barrier to resist movement of plunger 24 via ball detent 74. Referring to FIGS. 3 and 7A, in the second position, ball detent 74 is located adjacent first end wall 51 of circumferential slot 50. Referring to FIGS. 4 and 7B, in the third position, ball detent 74 is located adjacent second end wall 53 of circumferential slot 50.

Referring to FIG. 4, once plunger 24 is rotated counter-clockwise approximately 90 degrees to the third position, cross-pin 72 of plunger 24 abuts flange exterior surface 46. This arrangement locks plunger 24 in the position shown in FIG. 4, i.e., the abutting relationship between cross-pin 72 and flange exterior surface 46 prevents the force of the vacuum seal from pulling plunger 24 in the direction of arrow A (FIG. 2) and inadvertently releasing the vacuum seal on glenoid head 30. This arrangement also prevents a user from accidently pushing plunger 24 relative outer housing 22 in the direction of arrow A (FIG. 2). Additionally, the bottom wall of outer housing cylindrical body 40 that defines circumferential slot 50 provides a physical barrier to resist movement of plunger 24 within outer housing cavity 60 toward suction cup 26 via ball detent 74.

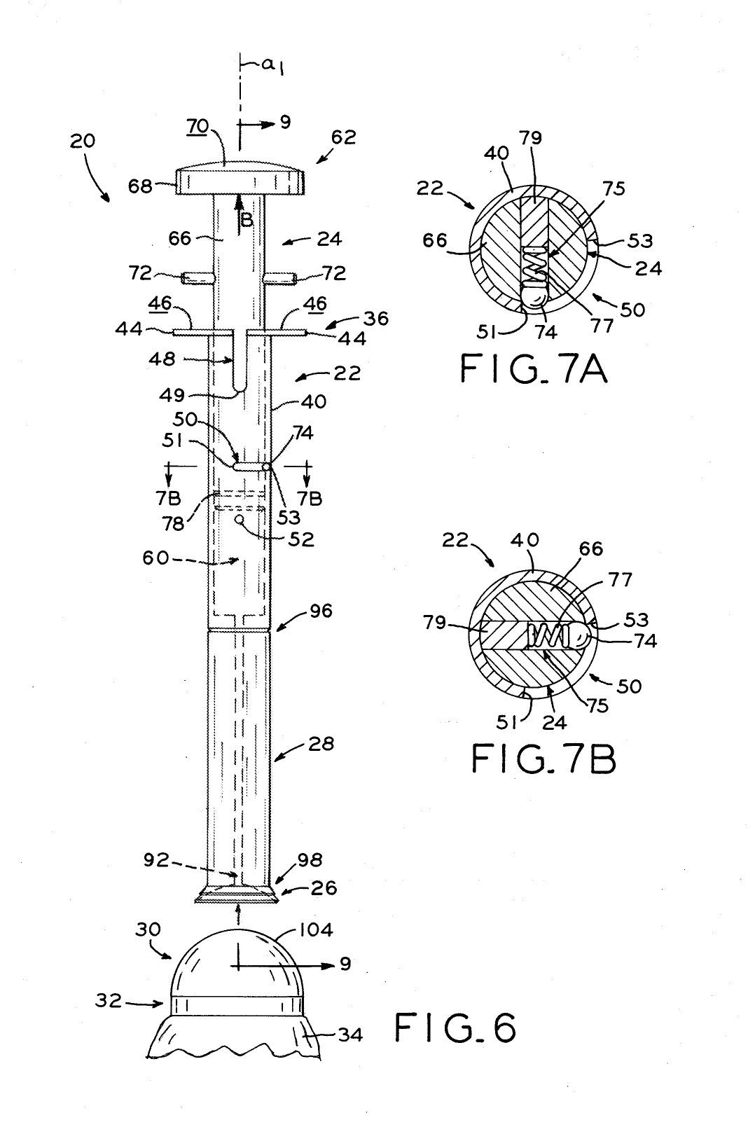

Once plunger 24 is rotated into the locked, third position shown in FIG. 4, the user may then use surgical instrument 20 to position glenoid head 30 relative to glenoid base plate 32 of a reverse shoulder prosthesis assembly. Referring to FIG. 5, in one embodiment, surgical instrument 20 with glenoid head 30 secured to suction cup 26 can be inserted in a surgical site along a linear path orthogonal to the orientation of glenoid base plate 32, i.e., plane bp₁ (FIG. 5) including glenoid head contacting surface 109 of glenoid base plate 32. In this manner, glenoid head 30 is positioned relative to glenoid base plate 32 such that planar end wall 106 of glenoid head 30 is in contact with glenoid head contacting surface 109 of glenoid base plate 32 as illustrated in FIG. 6.

As shown in FIG. 5, in one embodiment, central peg 108 of glenoid head 30 can be inserted within central aperture 110 of glenoid base plate 32 to facilitate the alignment between glenoid head 30 and glenoid base plate 32. Once glenoid head 30 is properly positioned relative to glenoid base plate 32, glenoid head 30 may be seated onto glenoid base plate 32 by providing an impaction force through surgical instrument 20 via impaction surface 70 of plunger impaction head 68. With plunger 24 in the locked, third position shown in FIGS. 4 and 5, the abutting relationship between cross-pin 72 and flange exterior surface 46 of outer housing 22 provides the resistance for transferring the impaction load through surgical instrument 20 while impacting glenoid head 30 onto glenoid base plate 32. The impaction load is transmitted from impaction surface 70 through body 66 of plunger 24 to cross-pin 72 to flange 44 to outer housing cylindrical body 40 to proximal end 38 of extending arm 42 of outer housing 22 to suction cup shoulder 94, suction cup 26 and glenoid head 30. In an embodiment utilizing sleeve 28, the impaction load is also transmitted from shoulder 58 of outer housing 22 to sleeve distal end 96 to sleeve 28 to annular sleeve proximal surface 99 to suction cup exterior surface 90 to suction cup 26 and to glenoid head 30. The locked, third position also allows an impaction force to be applied to impaction surface 70 while preventing the impaction force from moving plunger 24 relative outer housing 22 in the direction of arrow A (FIG. 2) and inadvertently releasing the vacuum seal on glenoid head 30. Impaction surface 70 is provided on an integrally formed, enlarged contoured impaction head 68 which also facilitates gripping and rotating plunger 24.

Referring now to FIG. 6, upon final seating of glenoid head 30 onto glenoid base plate 32, the user can disengage surgical instrument 20 from glenoid head 30 by breaking the vacuum seal. In one embodiment, the user can move plunger 24 along longitudinal axis a₁ of surgical instrument 20 from the third position (FIGS. 4 and 5) relative to outer housing 22 to a fourth position (FIG. 6) relative to outer housing 22. Plunger 24 can be moved by first holding outer housing 22 near flanges 44, for example, to hold outer housing 22 in a stationary position, and then pulling plunger 24 relative to outer housing 22 along the direction of arrow B (FIG. 3) until the sealing mechanism such as o-ring 78 (located adjacent proximal end 64 of plunger 24) traverses past air release aperture 52 (shown in FIGS. 6 and 9), thereby releasing the vacuum seal and unlocking glenoid head 30 from surgical instrument 20. Because the user can actively select and control when to disengage the vacuum seal, the glenoid head 30 can be selectively released from surgical instrument 20. The vacuum seal is released because once the sealing mechanism such as o-ring 78 (located adjacent proximal end 64 of plunger 24) is positioned such that air release aperture 52 is positioned between suction cup 26 and the sealing mechanism such as o-ring 78, as shown in FIG. 9, the air from outside of surgical instrument 20 can enter outer housing cavity 60 via air release aperture 52 to equalize the pressure within outer housing cavity 60 and release the vacuum seal. Referring to FIGS. 4 and 8, with cross-pin 72 abutting flange exterior surface 46, air release aperture 52 is not positioned between suction cup 26 and the sealing mechanism such as o-ring 78.

The user can also disengage surgical instrument 20 from glenoid head 30 by first rotating plunger 24 clock-wise, i.e., in a direction opposite arrow C (FIG. 4), from the third position (FIGS. 4 and 5) to the second position (FIG. 3). Next, plunger 24 is moved from the second position (FIG. 3) to the first position (FIG. 2) to break the vacuum seal and unlock glenoid head 30 from surgical instrument 20. After surgical instrument 20 is disengaged from glenoid head 30 by breaking the vacuum seal, surgical instrument 20 may then be removed from the surgical site.

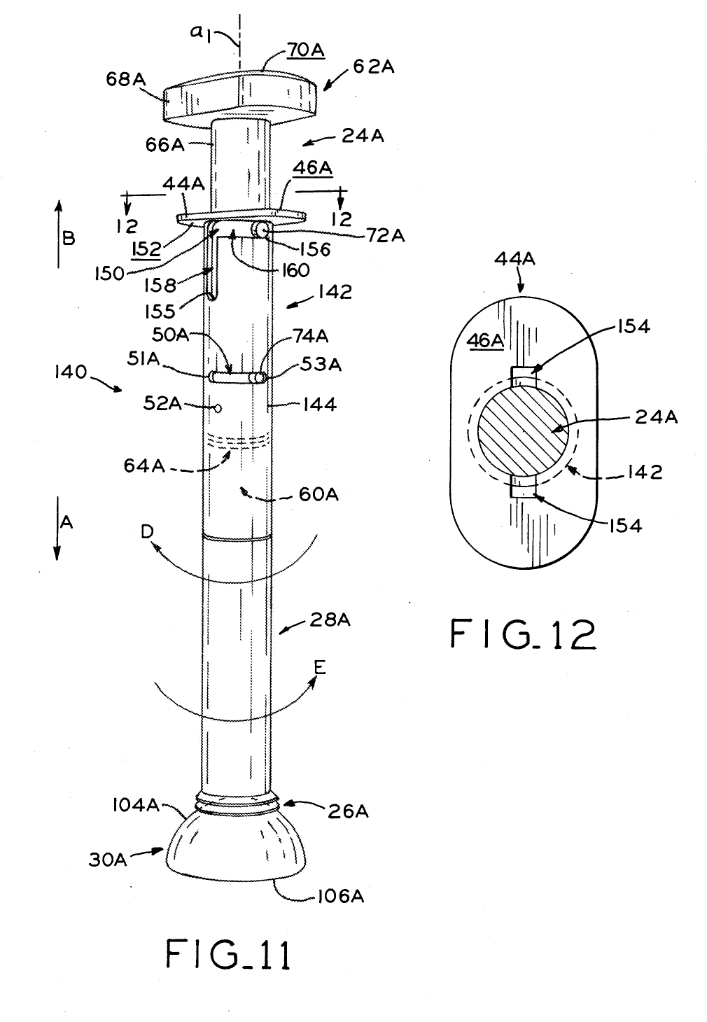

FIGS. 11 and 12 illustrate another exemplary embodiment. This embodiment of the present disclosure includes similar components to the embodiment illustrated in FIGS. 1-9, which are denoted by a reference number followed by the letter A. For the sake of brevity, these similar components and the similar steps of using surgical instrument 140 will not all be discussed in conjunction with the embodiment illustrated in FIGS. 11 and 12.

Referring to FIGS. 11 and 12, surgical instrument 140 includes outer housing 142, plunger 24A, suction cup 26A, and sleeve 28A. In this embodiment, outer housing 142 includes outer housing cylindrical body 144 having L-shaped slot 150 (another L-shaped slot is located on the opposite side of outer housing cylindrical body 144) extending from first end wall 155 to second end wall 156, and including longitudinal slot portion 158 and circumferential slot portion 160. In this embodiment, slots 150 replace longitudinal slots 48 (FIGS. 1-6). Referring to FIG. 11, slot 150 is located beneath flange 44A and includes longitudinal slot portion 158 extending longitudinally from first end wall 155 towards flange stopping surface 152 of flange 44A. At a position adjacent flange stopping surface 152 and beneath flange 44A, circumferential slot portion 160 extends along 90 degrees of body 144 and terminates at second end wall 156. Longitudinal slot portion 158 and circumferential slot portion 160 together form an L-shape. In this embodiment, flange 44A includes flange slots 154 (FIG. 12) spanning flange exterior surface 46A and flange stopping surface 152.

Referring to FIGS. 11 and 12, to assemble surgical instrument 140 a user such as a surgeon inserts proximal end 64A of plunger 24A into cavity 60A of outer housing cylindrical body 144 such that cross-pin 72A of plunger 24A is aligned with flange slots 154 of flange 44A. With cross-pin 72A of plunger 24A aligned with flange slots 154 of flange 44A, cross-pin 72A can be inserted through flange slots 154 and into respective circumferential slot portions 160 of slots 150. Next, plunger 24A can be rotated clockwise, i.e., in the direction of arrow D (FIG. 11), approximately 90 degrees such that cross-pin 72A is aligned with respective longitudinal slot portions 158 of slots 150. Rotation of plunger 24A is controlled by the engagement of cross-pin 72A with respective circumferential slot portions 160 of slots 150. Next, plunger 24A can be inserted further into cavity 60A of outer housing cylindrical body 144 until cross-pin 72A contacts respective first end walls 155 of longitudinal slot portions 158. In this manner, insertion of plunger 24A is controlled by the engagement of cross-pin 72A with respective longitudinal slot portions 158 of slots 150. This position of surgical instrument 140 corresponds to the initial or first position of plunger 24A relative to outer housing 142 (this is the same position as shown in FIG. 2).

Next, surgical instrument 140 can be positioned relative to glenoid head 30A and a user can releasably attach glenoid head 30A to surgical instrument 140 by forming a continuous seal between suction cup 26A and glenoid head 30A as previously discussed. Next, to provide the vacuum seal between surgical instrument 140 and glenoid head 30A as previously discussed, the user slowly moves plunger 24A along longitudinal axis a₁ in the direction of arrow B, from the first position, i.e., with cross-pin 72A in contact with respective first end walls 155 of longitudinal slot portions 158 of slots 150, until cross-pin 72A contacts flange stopping surface 152 of flange 44A. In this manner, movement of plunger 24A in the direction of arrow B is controlled by the engagement of cross-pin 72A with respective longitudinal slot portions 158 of slots 150. In this embodiment, flange stopping surface 152 acts as a stop mechanism to prevent a user from pulling plunger 24A out of cavity 60A of outer housing cylindrical body 144. In such a manner, a user can only pull plunger 24A along longitudinal axis a₁ in the direction of arrow B relative to outer housing 142 to form the vacuum seal between surgical instrument 140 and glenoid head 30A in a controlled manner, i.e., until cross-pin 72A contacts flange stopping surface 152 of flange 44A. In this embodiment, the engagement of cross-pin 72A of plunger 24A in respective longitudinal slot portions 158 of slots 150 provides proper alignment of plunger 24A relative to outer housing 142 and facilitates ball detent 74A engaging circumferential slot 50A.

Next, with cross-pin 72A of plunger 24A in contact with flange stopping surface 152 of flange 44A, the user can now rotate plunger 24A counter-clockwise, i.e., in the direction of arrow E, approximately 90 degrees relative to outer housing 142 until cross-pin 72A of plunger 24A contacts respective second end walls 156 of slots 150. Rotation of plunger 24A in the direction of arrow E is guided by the engagement of cross-pin 72A with respective circumferential slot portions 160 of slots 150 and the engagement of ball detent 74A with circumferential slot 50A. Further, the engagement of cross-pin 72A with respective circumferential slot portions 160 of slots 150 prevents plunger 24A from rotation relative to outer housing 142 beyond 90 degrees. Referring to FIG. 11, in this position, plunger 24A is in a locked position, i.e., the abutting relationship between cross-pin 72A and the walls of respective circumferential slot portions 160 of slots 150 and the abutting relationship between ball detent 74A and the walls of circumferential slot 50A prevent the force of the vacuum seal from pulling plunger 24A in the direction of arrow A and inadvertently releasing the vacuum seal on glenoid head 30A. In this position, a user may then use surgical instrument 140 to position glenoid head 30A relative to glenoid base plate 32 (FIG. 5) and glenoid head 30A may be seated onto glenoid base plate 32 (FIG. 5) by providing an impaction force through surgical instrument 140 via impaction surface 70A of plunger impaction head 68A as previously discussed and as shown in FIG. 5. Upon final seating of glenoid head 30A onto glenoid base plate 32 as shown in FIG. 6, the user can disengage surgical instrument 140 from glenoid head 30A and remove surgical instrument 140 from the surgical site as previously discussed.

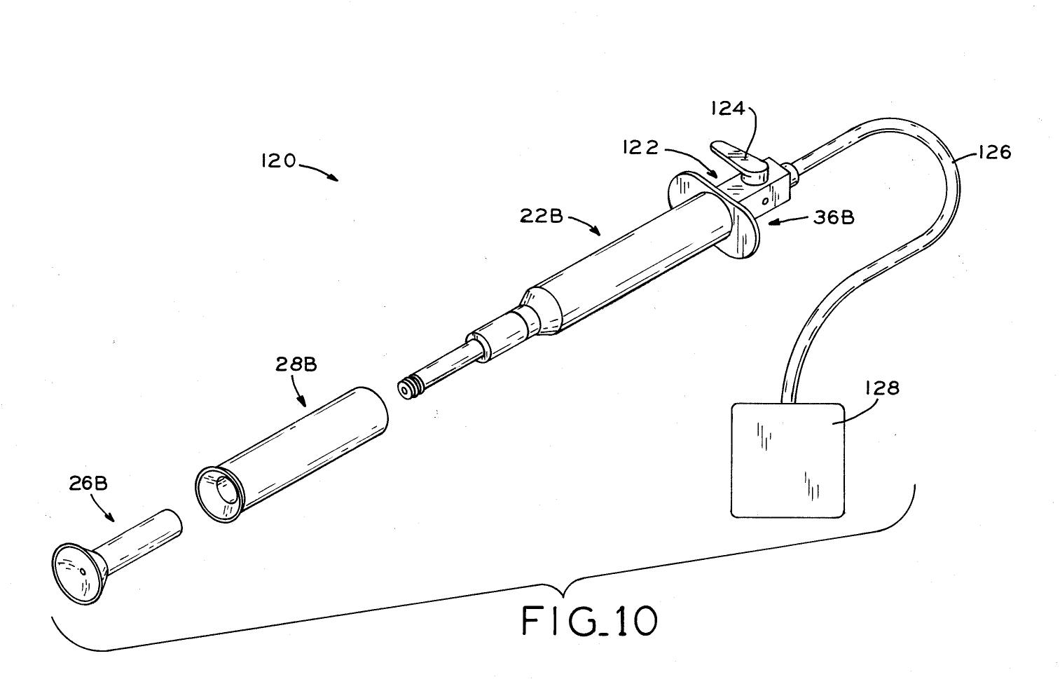

FIG. 10 illustrates another exemplary embodiment. The embodiment illustrated in FIG. 10 includes similar components to the embodiment illustrated in FIGS. 1-9, which are denoted by a reference number followed by the letter B. For the sake of brevity, the similar components will not all be discussed in conjunction with the alternate embodiment disclosed herein. Surgical instrument 120 includes outer housing 22B, suction cup 26B, and sleeve 28B. However, surgical instrument 120 does not include plunger 24 as illustrated in FIGS. 1-9. Similar to the exemplary embodiment illustrated in FIGS. 1-9, an active suction source is provided to surgical instrument 120. Referring to FIG. 10, the active suction source is provided to surgical instrument 120 by a vacuum source readily provided in a hospital environment such as vacuum source 128. Referring to FIG. 10, outer housing 22B does not include any slots or apertures. Instead, outer housing 22B includes nozzle 122 at distal end 36B. Nozzle 122 is used to removably connect hose 126 of vacuum source 128 to outer housing 22B. With hose 126 connected to nozzle 122, vacuum source 128 is in fluid communication with surgical instrument 120.

In this embodiment, vacuum source 128 is used to decrease the air pressure in outer housing 22B to create a vacuum seal. In this manner, with glenoid head 30 (shown in FIGS. 2-6) engaged to suction cup 26B of surgical instrument 120 as previously discussed, vacuum source 128 is used to secure suction cup 26B of surgical instrument 120 to glenoid head 30. In one embodiment, control valve 124 can be used to control the vacuum seal created by vacuum source 128 by opening and closing valve 124. This allows a user to select when to utilize the vacuum seal and when to disengage the vacuum seal. In one embodiment, control valve 124 can have an on position and an off position. With control valve 124 in the on position, vacuum source 128 is in fluid communication with surgical instrument 120, and with control valve 124 in the off position, vacuum source 128 is not in fluid communication with surgical instrument 120.

In another embodiment, it is envisioned that vacuum source 128 could be used to provide an additional suction source to the surgical instrument illustrated in FIGS. 1-9. An additional suction source could be used to enhance the vacuum seal provided between the surgical instrument and glenoid head 30.

While this disclosure has been described as having exemplary designs, the present disclosure can be further modified within the spirit and scope of this disclosure. This application is therefore intended to cover any variations, uses, or adaptations of the disclosure using its general principles. Further, this application is intended to cover such departures from the present disclosure as come within known or customary practice in the art to which this disclosure pertains and which fall within the limits of the appended claims.

What is claimed is:

1. A surgical instrument for securement to a prosthetic component so that the surgical instrument may be utilized to manipulate the prosthetic component relative to an anatomical structure, the surgical instrument comprising:
a prosthetic component contacting end sized and shaped relative to the prosthetic component such that said prosthetic component contacting end is capable of forming a continuous seal between the surgical instrument and the prosthetic component;
an outer housing having an outer housing proximal end, an outer housing distal end, and a wall defining an elongate aperture between said outer housing proximal end and said outer housing distal end, said elongate aperture in fluid communication with said prosthetic component contacting end;
a plunger having an outer periphery and a plunger central axis, said plunger sized for movement within said elongate aperture of said outer housing;
a seal interposed between said plunger and said wall of said outer housing, said seal sized relative to said plunger and said outer housing so that, with said prosthetic component contacting end forming said continuous seal with the prosthetic component, movement of said plunger within said elongate aperture of said outer housing away from said prosthetic component contacting end effectuates a vacuum seal between said prosthetic component contacting end and the prosthetic component; and
a lock connected to said outer housing and said plunger, said lock operable to selectively lock said plunger relative to said outer housing so that movement of said plunger within said elongate aperture of said outer housing toward said prosthetic component is prevented, whereby, with said lock locking said plunger relative to said outer housing so that movement of said plunger within said elongate aperture of said outer housing toward said prosthetic component is prevented, a force imparted to said plunger is transmitted to said prosthetic component contacting end via said lock and said outer housing.

2. The surgical instrument of claim 1, wherein said lock comprises:
a transverse protrusion extending from said outer periphery of said plunger transverse to said plunger central axis; and
a first slot formed in said wall of said outer housing, said first slot sized to receive said transverse protrusion, said first slot extending from said outer housing distal end toward said outer housing proximal end so that with said transverse protrusion withdrawn from said first slot and said plunger rotated relative to said outer housing so that said transverse protrusion is not aligned with said first slot, said outer housing distal end precludes movement of said transverse protrusion toward said outer housing proximal end to lock said plunger relative to said outer housing.

3. The surgical instrument of claim 2, wherein said wall of said outer housing includes an air release aperture formed therethrough, whereby, with said air release aperture positioned between said prosthetic component contacting end and said seal, said prosthetic component contacting end cannot effectuate said vacuum seal between said prosthetic component contacting end and the prosthetic component, said seal spaced a distance from said transverse protrusion such that, with said transverse protrusion abutting said outer housing distal end, said air release aperture is not positioned between said prosthetic component contacting end and said seal.

4. The surgical instrument of claim 2, further comprising:
a second lock connected to said outer housing and said plunger, said second lock operable to selectively lock said plunger relative to said outer housing so that movement of said plunger within said elongate aperture of said outer housing toward said prosthetic component is resisted.

5. The surgical instrument of claim 4, wherein said second lock comprises:
a detent slidably connected to said outer periphery of said plunger; and
a second slot formed in said wall of said outer housing, said second slot sized to receive said detent, with said transverse protrusion withdrawn from said first slot, said detent is engaged with said second slot.

6. The surgical instrument of claim 1, wherein said prosthetic component contacting end has a prosthetic component contacting end central axis, said outer housing has an outer housing central axis, said prosthetic component contacting end central axis aligned with said outer housing central axis and said plunger central axis.

7. The surgical instrument of claim 1, wherein said wall of said outer housing includes an air release aperture formed therethrough, whereby, with said air release aperture positioned between said prosthetic component contacting end and said seal, said prosthetic component contacting end cannot effectuate said vacuum seal between said prosthetic component contacting end and the prosthetic component.

8. The surgical instrument of claim 1, further comprising:
a support adjacent to said prosthetic component contacting end, said support extending to said continuous seal to support said prosthetic component contacting end.

9. The surgical instrument of claim 1, wherein said prosthetic component contacting end comprises a suction cup.

10. The surgical instrument of claim 1, wherein the prosthetic component comprises a glenoid head for use in a reverse shoulder arthroplasty and wherein said prosthetic component contacting end is sized and shaped relative to the glenoid head such that said prosthetic component contacting end is capable of forming said continuous seal between the surgical instrument and the prosthetic component.

11. The surgical instrument of claim 1, further comprising:
a second lock connected to said outer housing and said plunger, said second lock operable to selectively prevent said plunger from being removed from said elongate aperture of said outer housing.

12. A method of implanting a prosthetic component, comprising the steps of:
obtaining a surgical instrument comprising:
an active vacuum source; and
a prosthetic component contacting end sized and shaped relative to the prosthetic component such that the prosthetic component contacting end is capable of forming a continuous seal between the surgical instrument and the prosthetic component;
contacting the prosthetic component with the prosthetic component contacting end such that the continuous seal is formed;
actuating the active vacuum source to effectuate a vacuum seal between the prosthetic component contacting end of the surgical instrument and the prosthetic component to secure the prosthetic component to the surgical instrument;
positioning the prosthetic component, using the surgical instrument, until a desired position is achieved; and
imparting an impaction force to the prosthetic component through the surgical instrument.

13. The method of claim 12, wherein the surgical instrument further comprises a support abutting the prosthetic component contacting end.

14. The method of claim 12, wherein the surgical instrument further comprises an outer housing having an outer housing proximal end, an outer housing distal end, and a wall defining an elongate aperture between the outer housing proximal end and the outer housing distal end, the elongate aperture in fluid communication with the prosthetic component contacting end, a plunger having an outer periphery, the plunger sized for movement within the elongate aperture of the outer housing, and a seal interposed between the plunger and the wall of the outer housing, and wherein said step of actuating the active vacuum source comprises moving the plunger within the elongate aperture of the outer housing away from the prosthetic component contacting end.

15. The method of claim 14, wherein the surgical instrument further comprises a lock connected to the outer housing and the plunger, the lock operable to selectively lock the plunger relative to the outer housing so that movement of the plunger within the elongate aperture of the outer housing toward the prosthetic component is prevented, and before said step of imparting the impaction force, locking the plunger relative to the outer housing with the lock so that movement of the plunger within the elongate aperture of the outer housing toward the prosthetic component is prevented.

16. The method of claim 12, further comprising:
after said step of imparting the impaction force, disengaging the surgical instrument from the prosthetic component by breaking the vacuum seal.

17. A method of implanting a prosthetic component, comprising:
obtaining a surgical instrument, comprising:
a prosthetic component contacting end;
an outer housing having an outer housing proximal end, an outer housing distal end, and a wall defining an elongate aperture between the outer housing proximal end and the outer housing distal end, the elongate aperture in fluid communication with the prosthetic component contacting end;
a plunger having an outer periphery, the plunger sized for movement within the elongate aperture of the outer housing; and
a seal interposed between the plunger and the wall of the outer housing;
contacting the prosthetic component with the prosthetic component contacting end such that a continuous seal is formed;
after said step of contacting the prosthetic component, moving the plunger within the elongate aperture of the outer housing away from the prosthetic component contacting end to effectuate a vacuum seal between the prosthetic component contacting end of the surgical instrument and the prosthetic component to secure the prosthetic component to the surgical instrument;
positioning the prosthetic component, using the surgical instrument, until a desired position is achieved; and
disengaging the surgical instrument from the prosthetic component by breaking the vacuum seal.

18. The method of claim 17, wherein the prosthetic component comprises a glenoid head for use in a reverse shoulder arthroplasty and wherein said step of positioning the prosthetic component comprises positioning the glenoid head, using the surgical instrument, until a desired position relative to a glenoid is achieved.

19. The method of claim 17, further comprising:
before said step of disengaging the surgical instrument from the prosthetic component, imparting an impaction force to the prosthetic component through the surgical instrument.

20. The method of claim 19, wherein the surgical instrument further comprises a lock connected to the outer housing and the plunger, the lock operable to selectively lock the plunger relative to the outer housing so that movement of the plunger within the elongate aperture of the outer housing toward the prosthetic component is prevented, and before said step of imparting the impaction force, locking the plunger relative to the outer housing with the lock so that movement of the plunger within the elongate aperture of the outer housing toward the prosthetic component is prevented.
